(19) United States
(12) Patent Application Publication
(10) Pub. No.: US 2010/0211122 A1
(43) Pub. Date: Aug. 19, 2010

(54) HOLDER FOR SIMULTANEOUSLY APPLYING A GEL PACK AND A TRANSCUTANEOUS ELECTRICAL NERVE/MUSCLE STIMULATOR

(76) Inventor: Mark Hensley, Los Angeles, CA (US)

Correspondence Address: BERLINER & ASSOCIATES, 555 WEST FIFTH STREET, 31ST FLOOR, LOS ANGELES, CA, 90013, US

(21) Appl. No.: 12/665,464

(22) PCT Filed: Jun. 26, 2008
(86) PCT No.: PCT/US08/08017

§ 371 Date: Apr. 15, 2010

Related U.S. Application Data
(60) Provisional application No. 60/937,477, filed on Jun. 27, 2007.

(51) Int. Cl. A61N 1/02; A61F 7/02

(52) U.S. Cl. 607/3; 607/112; 607/111

(57) ABSTRACT

A device for application to a body part for the treatment of symptoms of injury or pain, comprising a holder for simultaneously applying (1) a thermal device capable of retaining hot or cold, and (2) a transcutaneous electrical nerve/muscle stimulator, whereby to allow the simultaneous application to a body part of transcutaneous electrical nerve/muscle stimulation and either hot or cold therapy. The holder can have a pocket for receiving the thermal device and a front surface bearing the nerve/muscle stimulator, for example, by hook and loop material, or adhesive, enabling the activatable surface of the nerve/muscle stimulator to be applied to the body part simultaneously with the hot or cold therapy of the gel pack in the pocket.

### CROSS-REFERENCE TO RELATED APPLICATIONS

This application claims the benefit of Provisional Patent Application No. 60/937,477, filed Jun. 27, 2007.

### FIELD OF THE INVENTION

The invention relates to a device for simultaneously applying a thermal device and a transcutaneous electrical nerve/muscle stimulator to a body part.

### BACKGROUND OF THE INVENTION

Transcutaneous electrical nerve/muscle stimulators for application to a body part are known and are commercially available, such as the Cosmo TENS™/EMS sold by Cosmo Health Inc., of West Covina, Calif. Such devices are secured to the front surfaces of pads of various sizes and shapes that have adhesive on their rear surfaces by which they are applied to a body part to provide spasm and pain relief and post surgical rehabilitation.

Schenck U.S. Patent Application Publication No. 2005/0043655, entitled “Back Support Device,” describes a back support that applies heat via a belt containing an electrical heater and, simultaneously, transcutaneous electrical nerve stimulation. Fruitman et al. U.S. Patent Application Publication No. 2006/0142816, entitled “Transcutaneous Electrical Nerve Stimulator With Hot Or Cold Thermal Application” describes a transcutaneous electrical nerve stimulator and electrode pad that is capable of delivering hot or cold thermal therapy for the relief of pain.

A serious drawback to the Schenck and Fruitman et al. devices is that they utilize electrical heaters or electrode pads, which result in significant expense, and are not readily adaptable to be worn on a variety of body parts.

### BRIEF SUMMARY OF THE INVENTION

The present invention enables the treatment of symptoms of injury or pain while overcoming the foregoing drawbacks. It simultaneously combines electrical stimulation with hot/cold therapy delivered in specially designed braces for all areas of the body to produce effective patient healing, strengthening and recovery. In contrast to the above-described electrode pad or electrical heater, the invention provides a holder for simultaneously accommodating (1) a thermal device for applying hot and/or cold therapy (such as a gel pack capable of retaining heat or cold, or other device), and (2) a transcutaneous electrical nerve/muscle stimulator, whereby to allow the simultaneous application to a body part of transcutaneous electrical nerve/muscle stimulation and either hot or cold therapy. The holder has a pocket for receiving the thermal device and a front surface bearing the nerve/muscle stimulator, for example, by hook and loop material enabling the activated surface of the nerve/muscle stimulator to be applied to the body part simultaneously with the hot or cold therapy of the thermal device in the pocket.

More particularly, the nerve/muscle stimulator has a front, activable, nerve/muscle stimulating surface and an opposite rear surface. Means are provided, such as complementary hook and loop material on opposing surfaces, for securing the rear surface of the nerve/muscle stimulator to the pocket front surface to enable the activated surface of the nerve/muscle stimulator to be applied to the body part simultaneously with the hot or cold therapy of the thermal device in the pocket. Preferably the nerve/muscle stimulator is of substantially smaller size than the holder, and the front surface of the holder and the rear surface of the nerve/muscle stimulator are of such respective sizes as to facilitate placement of the nerve/muscle stimulator at various places on the body part. In other embodiments the holder can be as small as the nerve/muscle stimulator, or smaller, depending on the particular body part to which the device will be applied. Thus, the holder can be of a size and shape to fit around one or more of the following body parts: arm; elbow; wrist; palm; finger; thigh; knee; lower leg; ankle; heel; foot arch; or toe. The nerve/muscle stimulator is of substantially smaller size than the holder. A thermal device (such as a gel pack) can be provided of a size to fit the pocket of the holder for these applications.

In a particular and specific embodiment, the holder is an elongate cloth brace having respective loop and hook material on its opposite ends for wrapping around a body part. The brace comprises: a thermal device capable of retaining heat or cold to apply hot or cold therapy to a body part; one or more transcutaneous electrical nerve/muscle stimulators having a front, activatable nerve/muscle stimulating surface and an opposite rear surface; a controller for controlling the electrical frequency of the transcutaneous electrical nerve/muscle stimulator; and one or more electrical wires connecting the controller to the transcutaneous electrical nerve/muscle stimulator.

A pocket is formed in the holder for containing the thermal device and has a front surface formed to be in contact with a body part. Loop or hook material is provided on the front surface of the holder and corresponding loop or hook material is provided on the rear surface of the nerve stimulator for securing the rear surface of the nerve stimulator to the pocket front surface to enable the activated surface of the nerve stimulator to be applied to the body part simultaneously with the hot or cold therapy of the thermal device in the pocket.

An additional pocket is formed in the holder for containing the controller and a flap is formed on the holder behind which can be placed the one or more electrical wires or wires from the controller to the transcutaneous electrical nerve stimulator.

In an alternative embodiment, one can apply a layer of adhesive to the rear side of the nerve/muscle stimulator to secure it to a plain pocket front surface.

### BRIEF DESCRIPTION OF THE DRAWINGS

For a more complete understanding of the present invention, reference is now made to the following descriptions taken in conjunction with the accompanying drawing, in which:

FIG. 1 is a plan schematic view of the front side of a holder in accordance with this invention;

FIG. 2 is a plan schematic view of the rear side of the holder of FIG. 1;

FIG. 3 is a cross-sectional view of the holder of FIG. 1, taken on line 3-3 of FIG. 1;

FIG. 4 is a cross-sectional view of an alternative embodiment using adhesive to secure the transcutaneous electrical nerve/muscle stimulator;

FIG. 5 is a front plan view of a transcutaneous electrical nerve/muscle stimulator used in this invention;

FIG. 6 is a rear plan view of the transcutaneous electrical nerve/muscle stimulator of FIG. 5; and

FIG. 7 is a rear plan view of an alternate embodiment of the transcutaneous electrical nerve/muscle stimulator of FIG. 5.

### DETAILED DESCRIPTION OF THE INVENTION

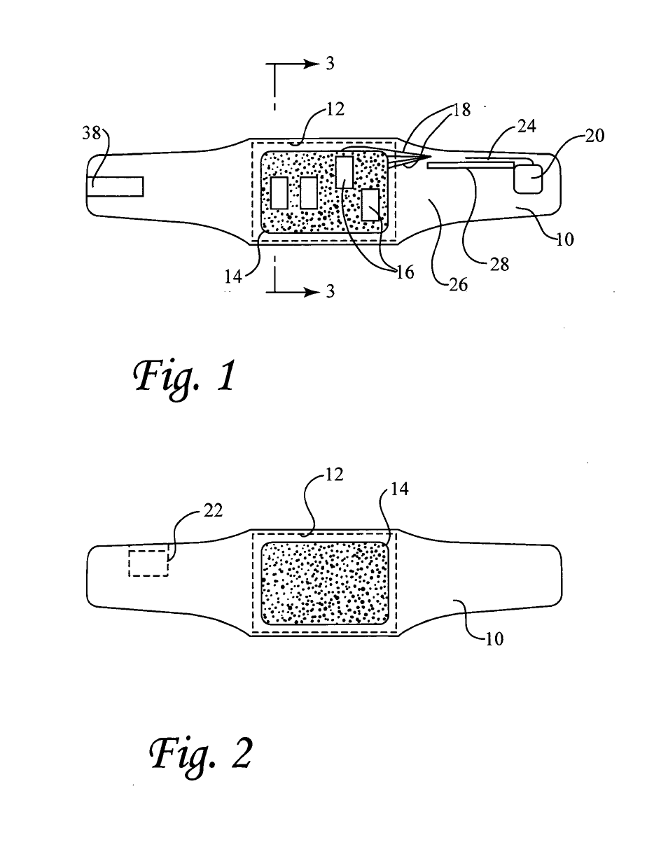

Referring to FIGS. 1 and 2, a holder 10 is shown as an elongate cloth for wrapping around a body part. The brace includes a pocket, shown in shadow at 12, formed to hold a gel pack, shown in phantom at 14, capable of retaining heat or cold to apply hot or cold therapy to a body part. Such gel packs are well known and commercially available in a variety of shapes and sizes. To the extent that a particular size gel pack is not commercially available, it can readily be manufactured using well known technology. For hot therapy, the gel pack can be heated, for example, by being placed in a microwave oven, to a desired temperature and will retain that heat for a substantial period of time. For cold therapy, the gel pack can be placed in a refrigerator, or in a freezer, depending on the degree of cold therapy desired.

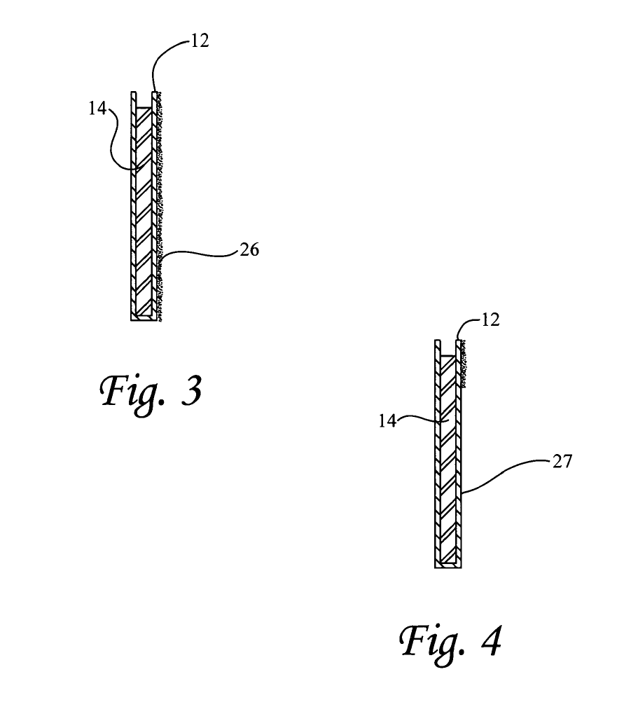

One or more transcutaneous electrical nerve/muscle stimulators, shown schematically at 16, are held onto the front surface of the holder 10. One or more electrical wires 18 for respective stimulator(s) 16 connect them to a controller 20 for controlling the electrical frequency of the stimulator, as known. A pocket, shown in shadow at 22, is formed on the holder 10 to contain the controller. A hook and loop flap connector 24 is formed on the front of the holder 10 and covers the wire(s) 18. Referring also to FIG. 3, in this embodiment, the entire front surface of the holder 10 carries a layer 26 of hook or loop material. It is preferred that the layer 26 be formed of loop material as it is softer on the skin. Correspondingly, the flap connector 24 is formed with a layer 28 of hook material on its open surface so that it can be folded over the electrical wire(s) 18 and connected to the loop material 26 on the front of the holder 10.

Referring to FIG. 4, in an alternate embodiment, the front surface 27 of the gel pack pocket holder 12 is smooth to receive adhesive as described below with respect to FIG. 7.

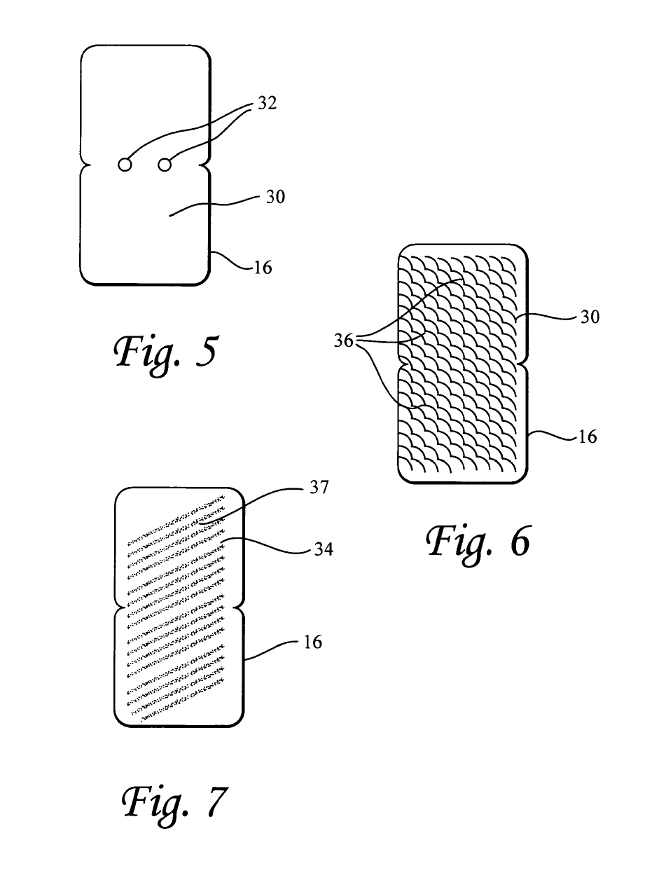

Referring to FIG. 5 the front side 30 of a transcutaneous electrical nerve/muscle stimulator 16 is shown with activatable electrodes 32 on its face to provide an activated nerve/muscle stimulating surface. FIG. 6 shows the rear side 34 of the transcutaneous electrical nerve/muscle stimulator 16 on which a layer 36 of loop or hook material is fixed, preferably hook material, corresponding to the material 26 on the front of the holder 10. By such means, one or more (four are shown in FIG. 1) transcutaneous electrical nerve/muscle stimulators can be placed in any desired position on the front surface of the gel pack pocket 12. As stated in the Background of the Invention, transcutaneous electrical nerve/muscle stimulators 16 are commercially available. Referred to by its initials as TENS, it is a battery powered device that transmits a mild electric impulse by the electrodes 32 through the skin to underlying nerve fibers and muscles to modify the perception of pain.

Referring again to FIG. 3, the pocket 12 containing the gel pack 14 has a front surface formed to be in contact with a body part. Loop or hook material is provided on the front surface of the holder and corresponding loop or hook material is provided on the rear surface 34 of the nerve/muscle stimulator 16 for securing the stimulator to the front surface of the pocket 12 to enable the activated electrodes 32 on the surface 30 of the stimulator to be applied to the body part simultaneously with the hot or cold therapy of the gel pack in the pocket.

Referring back to FIG. 1, a strip of hook material is fixed to one end of the front of the holder 10 permitting the holder to be affixed around a body part.

While the holder 10 has been illustrated with loop material 26 entirely over its front surface, the loop material can as well be limited to the front surface of the gel pack pocket 12. In that case a strip of loop material will be affixed to the rear surface of one end of the holder to connect to the strip of loop material 38 On the front surface of the other end of the holder 10.

FIG. 7 shows an alternative embodiment in which the holder has a smooth surface and a layer of adhesive 37 is applied to the rear surface of the transcutaneous electrical nerve/muscle stimulator used in this invention

The holder is preferably formed of cloth so that the heat or cold of the gel pack can be transmitted efficiently to the skin of the body part, but other materials, such as polyethylene or polypropylene polymer materials or other plastic material can be used. When using cloth, the pockets 12 and 22 and strip of hook material 38 can be fixed to the holder by sewing. Seam welding can be used when the holder is formed of plastic material.

While a particular form of holder has been shown, the holder can be of a size and shape to fit around one or more of the following body parts: arm; elbow; wrist; palm; finger; thigh; knee; lower leg; ankle; heel; foot arch; or toe. The nerve/muscle stimulator is of substantially smaller size than the holder. As indicated above, a gel pack can be provided of a size to fit the pocket of the holder for these applications.

Although the figures illustrate a gel pack, any thermal device can be used, and the invention encompasses the use of a thermal device that provides cold or enables a choice between cold and heat in combination with the nerve/muscle stimulator.

Moreover, although the present invention has been described in connection with the preferred embodiments, it is to be understood that modifications and variations may be utilized without departing from the principles and scope of the invention, as those skilled in the art will readily understand. Accordingly, such modifications may be practiced within the scope of the following claims.

1. A device for application to a body part for the treatment of symptoms of injury or pain, comprising a holder for simultaneously applying (1) a thermal device capable of applying cold or a choice of cold or heat, and (2) a transcutaneous electrical nerve/muscle stimulator, whereby to allow the simultaneous application to a body part of transcutaneous electrical nerve/muscle stimulation and either hot or cold therapy.

2. The device of claim 1 in which the thermal device is a gel pack capable of retaining heat or cold.

3. The device of claim 2 in which the holder has a pocket for receiving the gel pack, the pocket having a front surface formed to be in contact with a body part.

4. The device of claim 3 in which said nerve/muscle stimulator has a front, activatable nerve/muscle stimulating surface and an opposite rear surface, and including means for securing the rear surface of the nerve/muscle stimulator to said pocket front surface whereby to enable the activatable surface of the nerve/muscle stimulator to be applied to the body part simultaneously with the hot or cold therapy of the gel pack in the pocket.

5. The device of claim 4 in which the front surface of the holder and the rear surface of the nerve/muscle stimulator are of such respective sizes as to facilitate placement of the nerve/muscle stimulator at various places on the body part.

6. The device of claim 4 in which the rear surface of the nerve/muscle stimulator and the front surface of the gel pack pocket are joinable by complementary hook and loop material on said surfaces.

7. The device of claim 6 in which the loop material is on the front side of the gel pack pocket and the hook material is on the rear side of the nerve/muscle stimulator.

8. The device of claim 4 in which the rear surface of the nerve/muscle stimulator and the front surface of the gel pack pocket are joinable by adhesive.

9. The device of claim 8 in which the adhesive is carried by the rear surface of the nerve/muscle stimulator prior to being secured to the front surface of the gel pack pocket.

10. The device of claim 1 in which the holder is a brace.

11. The device of claim 10 in which the brace is elongate and formed with means at opposite ends for permitting the brace to be affixed around a body part.

12. The device of claim 10 in which the brace is of a size and shaped to fit around one or more of the following body parts:
(a) arm;
(b) elbow;
(c) wrist;
(d) palm;
(e) finger;
(f) thigh;
(g) knee;
(h) lower leg;
(i) ankle;
(m) heel;
(n) foot arch; or
(o) toe.

13. The device of claim 1 including a gel pack.

14. The device of claim 13 including a gel pack of a size to fit the pocket of the holder.

15. The device of claim 1 in which the nerve/muscle stimulator is of substantially smaller size than the holder.

16. The device of claim 1 in which the holder is formed of cloth.

17. The device of claim 1 including an additional pocket on the holder, the additional pocket being formed to contain a controller for controlling the electrical frequency of the transcutaneous electrical nerve/muscle stimulator.

18. The device of claim 17 including a flap on the holder behind which can be placed electrical wire or wires from the controller for the transcutaneous electrical nerve/muscle stimulator.

19. An elongate brace for wrapping around a body part for the treatment of symptoms of injury or pain, comprising:
a thermal device capable of retaining heat or cold to apply hot or cold therapy to a body part;
one or more transcutaneous electrical nerve/muscle stimulators having a front, activatable nerve/muscle stimulating surface and an opposite rear surface;
a controller for controlling the electrical frequency of the transcutaneous electrical nerve/muscle stimulator;
one or more electrical wires connecting the controller to the transcutaneous electrical nerve/muscle stimulator;
a pocket in the holder for containing the thermal device and having a front surface formed to be in contact with a body part;
means for securing the rear surface of the nerve/muscle stimulator to the pocket front surface whereby to enable the activated surface of the nerve/muscle stimulator to be applied to the body part simultaneously with the hot or cold therapy of the gel pack in the pocket;
an additional pocket in the holder for containing the controller;
a flap on the holder behind which can be placed the one or more electrical wires from the controller to the transcutaneous electrical nerve/muscle stimulator; and
respective loop and hook material on the opposite ends of the brace for permitting the brace to be affixed around a body part.

20. The brace of claim 19 wherein the securing means is selected from adhesive and complementary hooks and loops.
